(19) United States
(12) Patent Application Publication
(10) Pub. No.: US 2022/0125548 A1
(43) Pub. Date: Apr. 28, 2022

(54) SURGICAL HEAD HOLDING DEVICE

(71) Applicant: Olaf STORZ, Tuttlingen (DE)

(72) Inventor: Olaf STORZ, Tuttlingen (DE)

(21) Appl. No.: 17/280,004

(22) PCT Filed: Nov. 7, 2019
(86) PCT No.: PCT/EP2019/080585

§ 371 Date: Mar. 25, 2021

(30) Foreign Application Priority Data
Nov. 7, 2018 (DE) 20 2018 005 143.8
Jan. 25, 2019 (DE) 10 2019 101 947.8
Jan. 28, 2019 (DE) 10 2019 102 047.6

(51) Int. Cl. A61B 90/14; A61B 90/57

(52) U.S. Cl. CPC A61B 90/14; A61B 90/57

(57) ABSTRACT

The present invention relates to a surgical head holding device (1000) for use in a medical treatment. The head holding device (1000) encompasses hereby a head clamp (100). The head clamp comprises a first arm (101) and a second arm (103), wherein the first arm (101) as well as the second arm (103) comprise each a pin (101s, 103s), in order to receive or to clamp the head therein. Furthermore, the head holding device (1000) comprises a first bar (401) and a first connection arrangement (301) for connecting the first bar (401) to the head clamp (100).

The present invention relates to a surgical head holding device according to claim 1 and a first bar according to claim 10.

During a series of surgical operations in the head area, it is necessary to hold the patient's head in the desired position. In practice, for example head clamps are used for this purpose. They are stationary fixed, e.g. on the operating table, and comprise pins that apply pressure to the bony head from different sides of the head in order to clamp the head between them.

It is an object of the present invention to provide another surgical head holding device and a bar for such a head holding device.

The object according to the present invention is achieved by a surgical head holding device (also in short: head holding device) with the features of claim 1 and the bar (also referred to herein as the first bar) with the features of claim 10.

The present invention, therefore, proposes a surgical head holding device for use in a medical treatment.

The head holding device comprises a head clamp with a first arm and a second arm. Both the first arm and the second arm have, if provided, at least one pin each. Optionally, the pins and/or other elements for contacting and holding the head are provided. The sections carrying the pins or the contact elements, for example the arms, are arranged such that a patient's head may be received or clamped between them.

Optionally, one or both of the arms comprises a section which respectively extends horizontally in sections and ascends therefrom in sections. In this, the horizontal sections of the two arms may be adjustably connected to each other, for example by a ratchet or a toothed rail.

Furthermore, the head holding device optionally comprises a first bar and optionally a first connection arrangement or connection device for connecting the first bar to the head clamp.

The connection may be done directly, e.g. the first connection arrangement may be connected directly to a section of one of the arms, or indirectly, e.g. the first connection arrangement may itself be connected to a section of a clamp or of a clamping device connected to the arm, which may optionally also be provided.

According to the present invention, a bar (here: first bar) is further proposed which comprises, or is connected to, preferably at an end section thereof, a rail (alternatively: plate) extending radially and/or perpendicular to the longitudinal axis of the first bar, for the connection to a head clamp for a surgical head holding device, for instance for the head holding device according to the present invention. Its design, features or elements may respectively be designed as disclosed herein.

Embodiments according to the present invention may comprise one or more of the aforementioned or following features in any combination, unless the person skilled in the art recognizes the specific embodiment as technically impossible.

In all of the following statements, the use of the expression “may be” or “may have” and so on, is to be understood synonymously with “preferably is” or “preferably has,” and so on respectively, and is intended to illustrate an embodiment according to the present invention.

Whenever numerical words are mentioned herein, the person skilled in the art shall recognize or understand them as indications of numerical lower limits. Unless it leads the person skilled in the art to an evident contradiction, the person skilled in the art shall comprehend for example the specification of “one” as encompassing “at least one”. This understanding is also equally encompassed by the present invention as the interpretation that a numerical word, for example, “one” may alternatively mean “exactly one”, wherever this is evidently technically possible for the person skilled in the art. Both understandings are encompassed by the present invention and apply to all numerical words used herein.

Whenever spatial information such as “top”, “bottom”, “horizontal”, etc. are made herein, they are preferably to be understood to refer to the intended use of the subject-matter of the present invention. In case of doubt, they refer to the representation in the figures.

Advantageous developments of the present invention are each subject-matter of the dependent claims and embodiments.

Whenever an embodiment is mentioned herein, it thus represents an exemplary embodiment according to the present invention.

In some embodiments, the first bar comprises, preferably at an end section thereof, a rail extending radially and/or perpendicular to the longitudinal axis of the first bar.

The longitudinal axis of the first bar may be the longitudinal axis of its elongated section or of the main body, optionally that section which, as intended, is received in a fixing device, e.g. the first connection arrangement.

The rail preferably extends radially in both directions, thus, in at least one view, it preferably protrudes both to the left and to the right over or beyond the bar or its elongated base body.

The rail is preferably flat on its top and/or bottom surface. Elements such as screws, hooks, clamps, etc. protruding over the main plane of the top and/or of the bottom surface are preferably not provided, not part of the rail and/or not connected to the rail.

The rail is preferably longer than it is wide. It may be a plate or have a strip shape.

The rail is preferably perforated (through holes or blind holes).

Such holes or openings may comprise an internal thread.

The first bar is preferably welded to a threaded ring, which itself is welded to a threaded pin, and the latter may be welded and ground to the plate.

The rail closes the first bar preferably upwards or to one end of the bar.

In some embodiments, the head holding device further comprises at least one bracket, and a second connection arrangement configured for connecting the bracket to the rail. Encompassed by the present invention is also more than one bracket. The connection arrangements by which the optionally several brackets are connected to the rail may be of the same kind or different from each other.

The first and the second connection arrangement differ optionally from each other, in particular in the number of rotation axes, degrees of freedom, through-openings, etc. defined by them.

Thus, in some embodiments, either the first connection arrangement or the second connection arrangement is designed to define exactly a first axis of rotation and to enable a bar or bracket received therein to rotate exclusively about this first axis of rotation by this connection arrangement (each with respect to, for example, one stationary point or section, e.g. the arm, a clamp or the rail, to which the connection arrangement is itself attached).

In this, the other of these two connection arrangements is designed to define at least, or exactly, one second axis of rotation and a third axis of rotation and to enable a bar or bracket received therein to rotate about at least, or exactly, both the second axis of rotation and the third axis of rotation by this connecting arrangement.

In certain embodiments, the head holding device further comprises a second bar and also a third connection arrangement. The third connection arrangement is configured to receive the second bar together with the first bar, preferably releasably, preferably shiftable therein, preferably lockable, within it. A locking mechanism such as a clamping screw or the like may be provided.

The third connection arrangement is optionally identical in construction to the first connection arrangement.

The second bar optionally comprises, at least in sections, the same diameter and/or the same profile as the first bar.

In some embodiments, the third connection arrangement is configured to define exactly one fourth axis of rotation and to enable a bar received therein to rotate exclusively about the fourth axis of rotation by this connection arrangement.

The aforementioned axes of rotation may be parallel, skewed, intersecting, identical to each other.

For each of the connection arrangements mentioned herein, it may apply that the bar received by it may be fixed in several angular positions, preferably continuously, optionally at least within a predetermined angular range encompassing at least 45°.

In several embodiments, the first connection arrangement comprises a clamp or is connected thereto. The clamp serves to connect the first connection arrangement to the first arm, preferably to the section of the first arm carrying the at least one pin, e.g. to the ascending section of the arm.

In some embodiments, the clamp is designed to be releasably connectable to the arm.

In some embodiments, the clamp is designed to be shiftable along the first arm, e.g. in its longitudinal direction.

In several embodiments, the clamp comprises a plurality of interfaces, e.g. gear rims or dog clutches or dog couplings. These may be arranged in order to enable instruments or accessories to be coupled to the clamp, preferably from more than only one direction, and in their entirety preferably in more than one plane. For this purpose, they are provided, for example, at sections of the clamp which are provided at an angle to each other and/or not in the same plane.

In specific embodiments, although the clamp comprises clamping screw or threaded screw, it optionally does not comprise one or not exactly one claw. Preferably, the clamp does not have a claw, which is displaced or moved in the direction in which the clamping screw or threaded screw is also displaced for its use.

In certain embodiments according to the present invention, the head holding device comprises at least one section which is made of aluminum, titanium, stainless steel, plastic, carbon, composite material, a fiber-reinforced material or a combination thereof or comprises one of the materials mentioned here.

In some embodiments, the ascending section of at least one arm does not comprise a protruding rail.

For the first and the third connecting arrangement, it may apply that they can be moved or offset along the bars or projections of the clamp inserted into them. In this or therethrough, the position of this connection arrangement is adjusted relative to the bar inserted into them, or vice versa.

For the first connection arrangement and/or the third connection arrangement it may apply that they, respectively, comprise two or exactly two through-openings into which elements to be connected to each other, such as bars, protrusions or the like, may be inserted in order to be fixed therein. Clamping screws for the releasable fixing may be guided through screw passages likewise provided.

In some embodiments, any elements of the head clamping device are radiolucent or X-ray permeable.

In some embodiments, the first connection arrangement and the second connection arrangement do not define a common shifting axis of a bar received by both of these connection arrangements.

In some embodiments, the first connection arrangement comprises a bar, the second does not comprise the same bar.

In some embodiments, at least one connection arrangement, optionally more than one, comprises two through-holes each such that at least one of the bars referred to herein may be, or is, received in one of the through-holes so as to be slidable along the through-holes, lockable therein, or clampable therein.

In some embodiments, at least one of the bars comprises a radially protruding pin, projection or the like which prevents an undesired release of the bar from a connection arrangement in at least one direction, usually in its longitudinal direction. This may depend on the rotation position of the bar within the connection arrangement. For example, a groove or slot may be provided in the connection arrangement that allows the pin to pass therethrough depending on the rotational position of the bar. If the groove and pin overlap, the bar may be pulled out of the connection arrangement. If they do not overlap, for example because the bar has been rotated further by a few angular degrees, the pin cannot enter the groove for the bar to be pulled out. Consequently, it is not possible to pull the bar out in this rotational position thereof.

In some embodiments, the bracket and/or the second bar is curved in a cutting plane transverse to at least one cross-section thereof.

In some embodiments, the bracket and/or the second bar is not closed to form a ring or any other circumferential structure.

In some embodiments, the bracket and/or the second bar comprises a circular or ring-shaped cross-section.

In some embodiments, the bracket in the second connection arrangement is not arranged rotatably relative to the latter. Said bracket is, optionally, not rotatably arranged about a longitudinal axis thereof or about a section thereof in the second connection arrangement.

In some embodiments, the bracket and/or the second bar is at least partially hollow, in others it is not hollow in any section.

In several embodiments, the first connection arrangement comprises a locking screw or a clamping screw, the longitudinal axis of which is, during use of the object according to the present invention, vertical to the longitudinal direction of the horizontal arms of the head holding device.

In several embodiments, the second connection arrangement comprises a locking screw or a clamping screw, the longitudinal axis of which is perpendicular to the rail and/or parallel to the longitudinal axis of the first bar.

In some embodiments, the second connection arrangement is screwed to the rail, preferably from below, e.g. by its locking screw or clamping screw.

In some embodiments, the bracket in the second connection arrangement is screwed to the rail, preferably from below, e.g. by its locking screw or clamping screw.

In some embodiments, the second connection arrangement is connected to the rail in a rotation-proof manner, for example by welding, by suitable plug connections, by using an anti-rotation protection (for example in the form of one or several radially inserted pins), or the like.

In several embodiments, the rail is welded to the first bar, integrally manufactured or otherwise connected in a rotationally fixed, non-releasable and/or form-fit and/or force-fit manner. In other embodiments, the rail may be provided relative to the bar or to its main body in pivotable and/or shiftable manner. Such a movement may optionally be allowed or prevented by a fixing device. A fixing device may be e.g. a clamping device.

In some embodiments, the rail sits on the end of the first bar and/or covers its elongated body or main body.

In some embodiments, the longitudinal axis of the first bar passes or extends through the rail.

In some embodiments, the head holding device comprises at least one rail as well as at least one bracket.

In several embodiments, the rail is wider than it is thick or high. For example, it may be 2 cm to 10 cm wide or more, and its thickness may be, for example, between 0.5 cm to 2 cm or more.

The length of the rail may preferably be between 10 cm and 40 cm.

Preferably, the rail does not bear any pins for contacting the bony head.

In some embodiments, the rail comprises one or several through-openings and/or one or several blind openings. Such openings may comprise threaded inserts or threaded bores.

Such openings may be accessible from an upper side, lower side, front side and/or from lateral limitations surfaces.

The aforementioned openings, through-openings, bores, threaded bores, etc. may be used to attach further instruments, target devices or gel pads, for example as a hand rest, to the rail.

In some embodiments, the second connection arrangement does not extend, on a side, e.g. the one that is the upper side during use, beyond the rail. This may prevent the surgeon supporting himself on the rail from getting stuck thereon for example with his gloves or the sleeves of his surgical gown.

In some embodiments, at least any one of the connection arrangements comprises two clamping sections, which each surround a through-opening and which may be reversibly limited in diameter, preferably by only one locking screw or clamping screw, for clamping e.g. a bar. Both clamping sections may preferably be provided to be rotatable relative to each other. For example a gear rim, may for this purpose be provided between them. The rotatability relative to each other may optionally also be canceled and/or allowed again using the locking screw or the clamping screw.

In some embodiments, a bar is a rod, a tube or the like.

In some embodiments, at least one of the arms, especially with a view of its front side, comprises a section, e.g. the ascending section, with a profile designed in the form of a double T or an H. With this, a middle section is delimited in its longitudinal course by edge webs.

The edge webs may protrude above a level of the middle section, the middle section may be closed, that is, not being a through-opening or not consisting of through-opening(s).

Optionally, a section is provided in which the width of the middle section is constant. This section is optionally at least 1, 2 or 3 cm long.

In some embodiments, the bracket is mounted or fastened on one side or only on one side, in particular on a longitudinal end area of the bracket. The optional bearing may be, for example, a slide bearing. The optionally tubular bracket may thus be inserted into a sleeve-shaped opening of the second connection arrangement. The range of movement of the bracket may be limited in the slide bearing to rotation about exactly one axis and/or to translation along exactly one axis.

In several embodiments, the bracket is not supported on both sides, in particular not at its two longitudinal end areas, nor connected to the second connection arrangement or to the rail.

In some embodiments, the first bar is supported by a slide bearing. In the slide bearing, the first bar may be restricted to a direction of rotation about an axis and/or a direction of translation along this axis. The first bar may be arranged rotatably and/or shiftably in a sleeve-shaped opening in the form of a slide bearing, in particular by a tube section.

In some embodiments, the first bar is not supported in the first connection arrangement by a ball joint.

In several embodiments, the bracket may have a free end. A free end may mean that the bracket is not attached and/or is not supported at the free end.

One or several of the above-mentioned and of the following advantages may be achieved by embodiments according to the present invention.

An advantage of the present invention is that the surgeon has a plurality of degrees of freedom by using the connection arrangement for setting his instruments which are fixed to the head holding device.

In addition, when using a first and a second bar, he is optionally granted rotation possibilities in at least or in exactly three degrees of freedom. The larger number of degrees of freedom is preferably shifted closer to the connection of the attachment comprising the bars and brackets to the arm, which may have advantages in terms of stability and strength of the structure.

Furthermore, the rail (or rails, see the following figures) mentioned herein may be of particular use to him in his work, as it enables him to support his arm, hand and the like.

The head holding device according to the present invention may allow that he does not get stuck on locks, handles, screw ends and the like as in embodiments described herein.

In addition, due to the design of the rail and/or its connection to one of the arms of the head holding device, it may be possible to attach a multitude/plurality of brackets on the rail next to each other. These may extend in one and the same direction from the rail, e.g. into the space between the arms (e.g. instruments) or may be facing outwards (e.g. navigation; imaging, etc.). The brackets may be attached to the top and/or to the bottom of the rail, they may be rotatable and thus pivotably connected to the rail. In this, the plate shape of some embodiments allows a broad-based support of the instrument or accessory (for example by simultaneous connection in openings of the rail that are relatively far apart from each other), which may increase the stability.

Since the rail is preferably flat and long, its dimensions may be chosen to comparatively generous, so that it offers space for coupling a large number of interfaces or instruments etc. without its weight increasing to an alarming extent.

In some embodiments, the first bar is optionally not positioned centrally under the rail. This may provide greater flexibility by rotating the first bar, sometimes turning the less protruding leg of the rail into or further into the space between the arms and sometimes turning the more protruding leg. Should more space be needed between the attachments attached to the arms during surgery, this may allow to create said space by rotating the shorter legs of the rail or of the oppositely-lying rails (i.e. those with less projection over the first bar) into this space, but not the longer legs. A reverse handling may also be considered, should instruments have to reach further into the space between the arms. In this case, the longer leg can be pivoted into the space. Overall, the respective rail, if the rail optionally has legs of unequal length, it allows a larger working area and/or working radius.

In some embodiments, target devices and/or other instruments may be attached to the rail. For this purpose, the above-mentioned openings may be used, alternatively or in addition, the rail may have one or more interfaces on at least one surface thereof (top, bottom, front, or any other), such as additional gear rims.

In several embodiments, the rail may serve as a support, for example a hand rest. A gel pad may optionally be attached or is attached.

In some embodiments, the brackets are compatible with commercially available clamps (e.g. Leyla, Greenberg systems) due to their design (e.g. the thin bracket with a round cross-section). This may advantageously save costs for the development of new clamps.

Advantageously, the first bar may be positioned at any angle to the fixed arms.

Advantageously, the attachment and/or navigation connections may be attached to the clamp. This offers considerable freedom and flexibility when attaching additional instruments.

The connection arrangement allows advantageously to attach a plurality of additional instruments and navigation connections.

Advantageously, the rail is secured against accidental falling out by the falling out-protection of or via or through the first bar. The same applies to further elements like the bracket or the second bar.

The attachment (bar with rail, with or without bracket, possibly with clamp) may advantageously be easily released from the respective arm using the proposed clamp connections, in particular by releasing only one main clamp (see reference number 201 in the figures), advantageously without a tool.

The present invention is exemplarily described below based on the accompanying, partly simplified figures, in which identical reference numerals refer to the same or to similar components. The following applies in the figures:

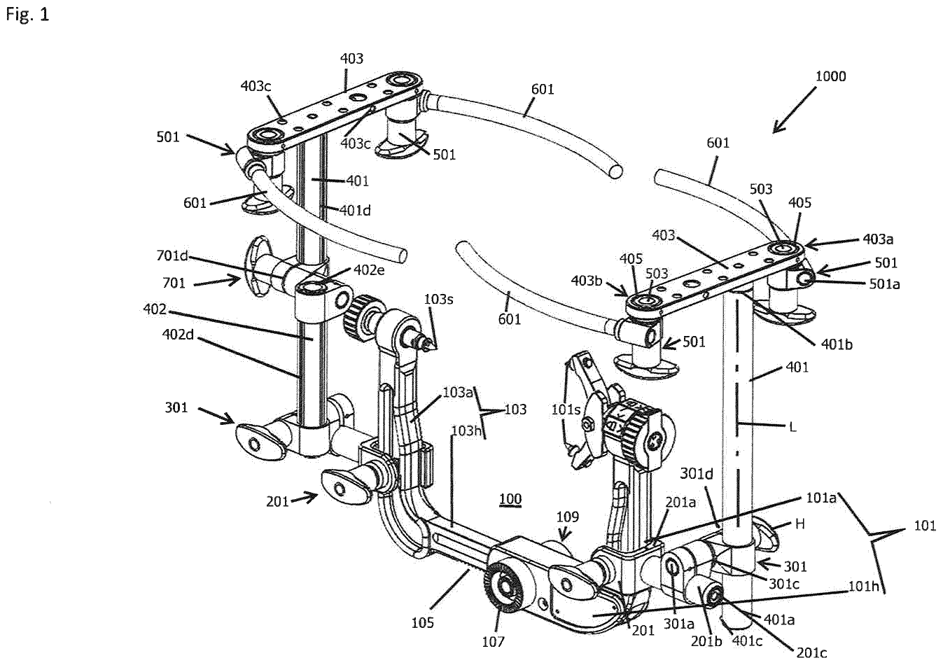

FIG. 1 shows a surgical head holding device according to the present invention in an exemplary first embodiment in a perspective view from the top right;

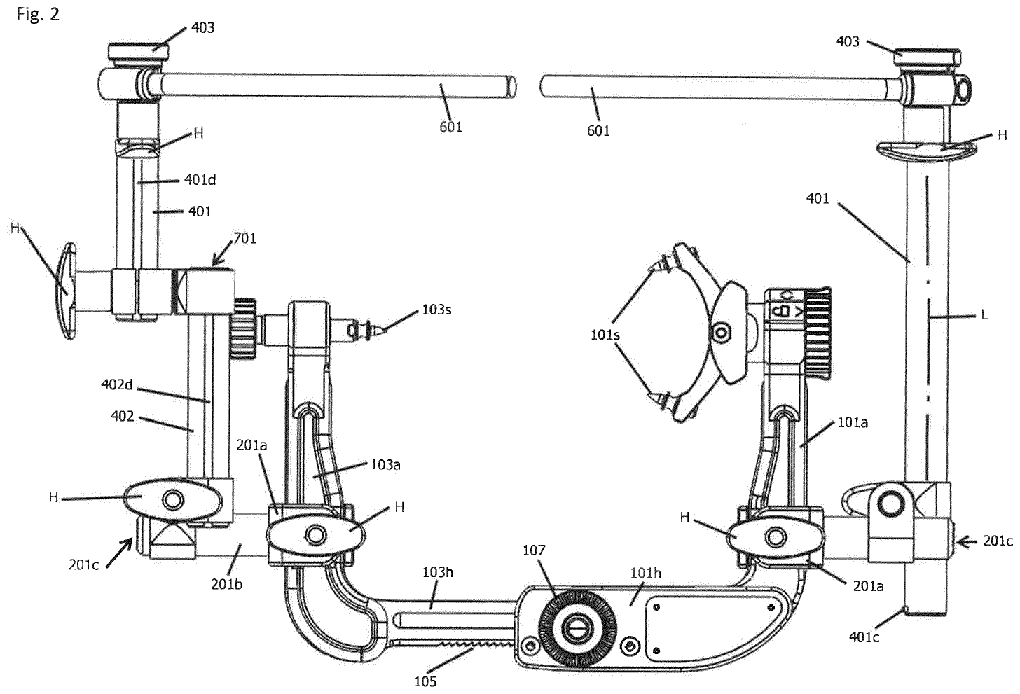

FIG. 2 shows sections of the surgical head holding device of FIG. 1 in side view;

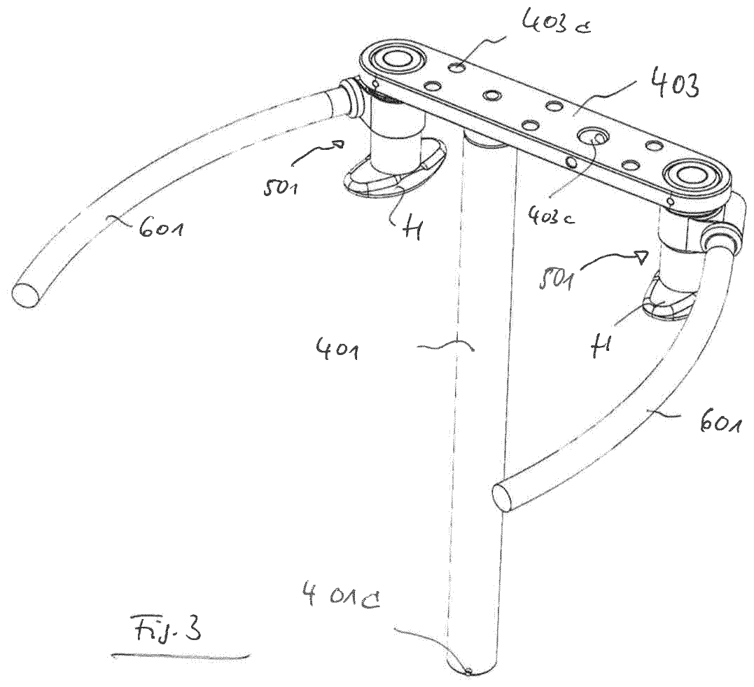

FIG. 3 shows elements shown in FIG. 1 in perspective;

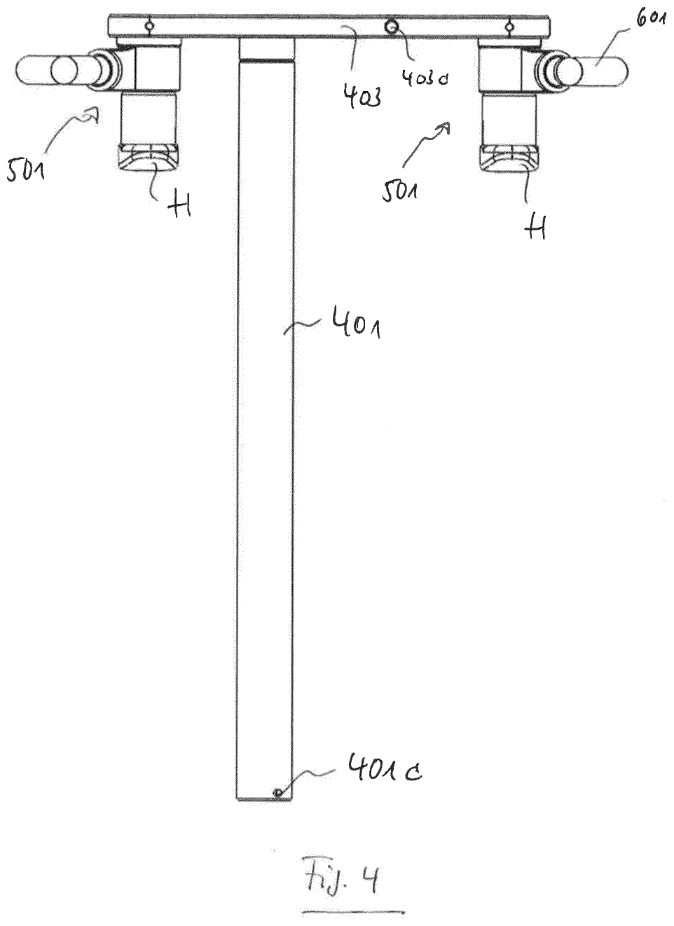

FIG. 4 shows the element shown in FIG. 3 from the front;

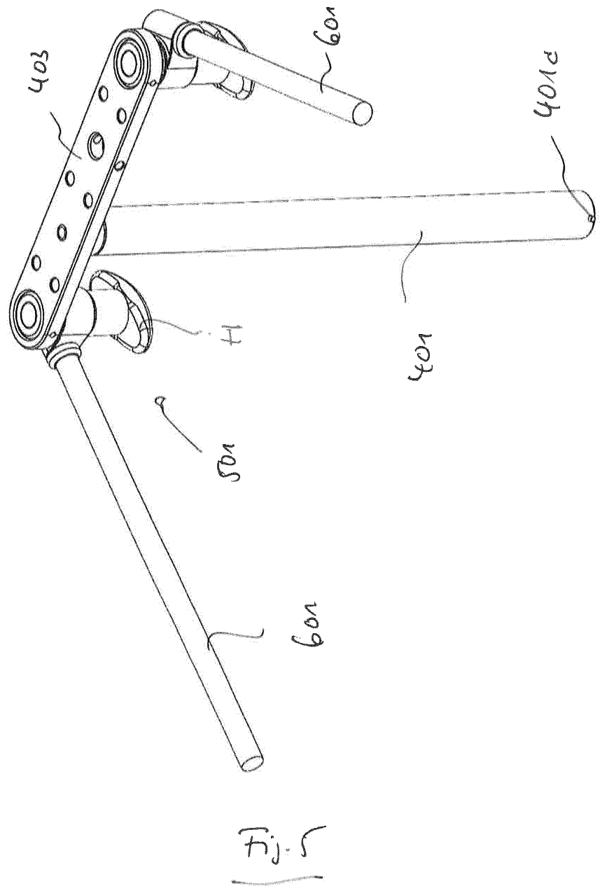

FIG. 5 shows an alternative embodiment of the element shown in FIG. 3;

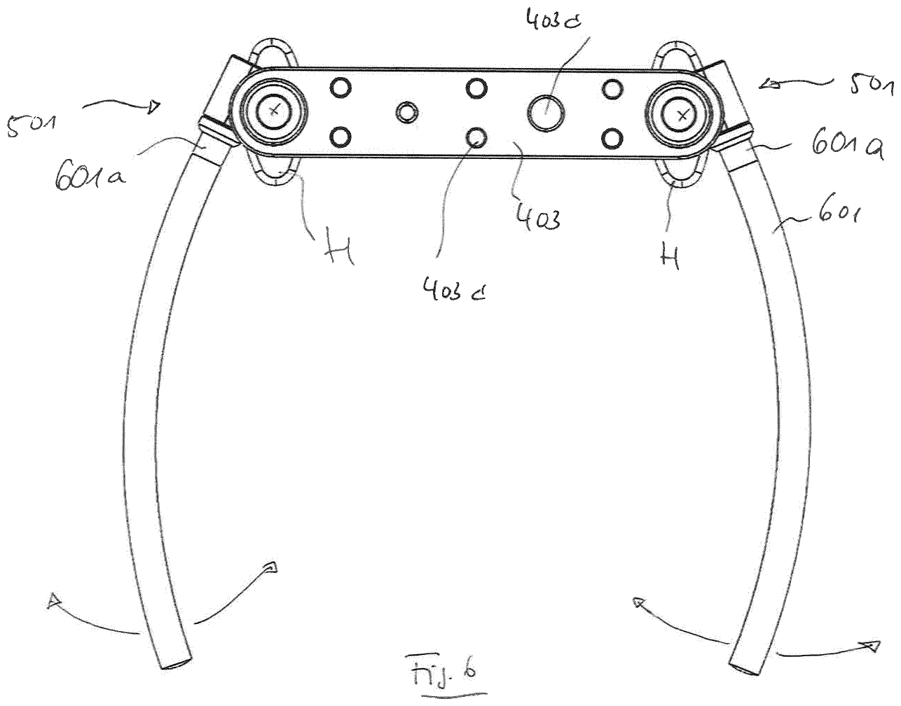

FIG. 6 shows the elements shown in FIG. 3 from above;

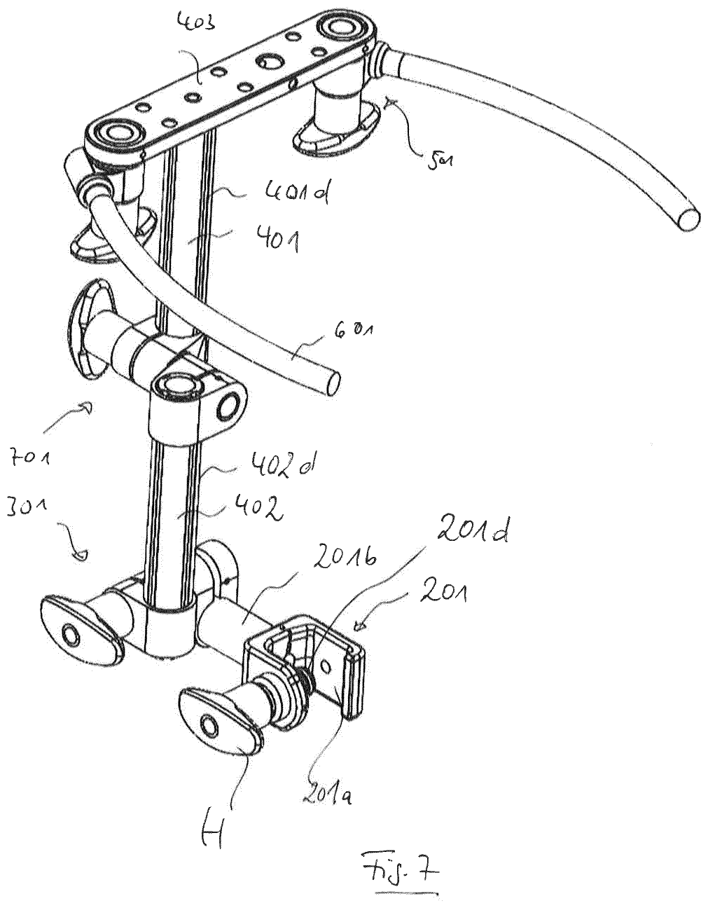

FIG. 7 shows the structure connected to the second arm in FIG. 1 in perspective;

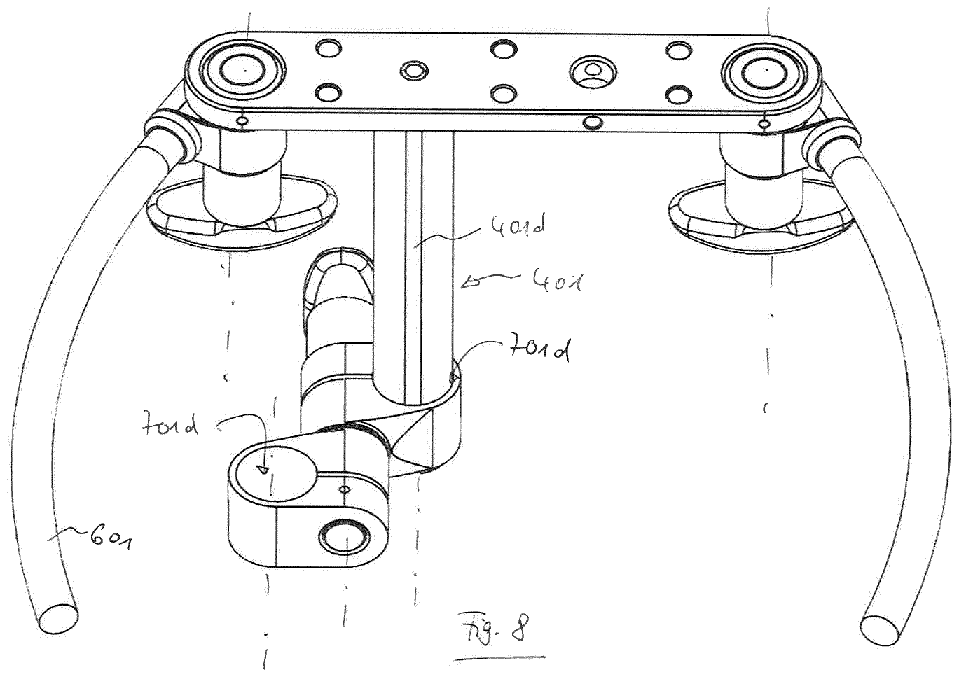

FIG. 8 shows sections of the structure shown in FIG. 7 from the front and slightly from above;

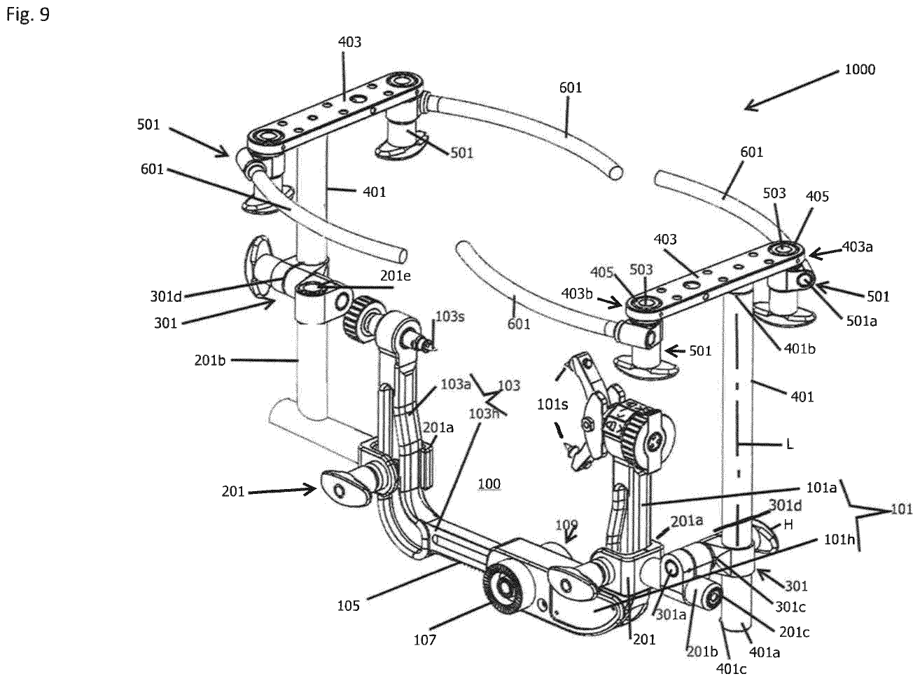

FIG. 9 shows a surgical head holding device according to the present invention in an exemplary second embodiment in perspective view from the top right;

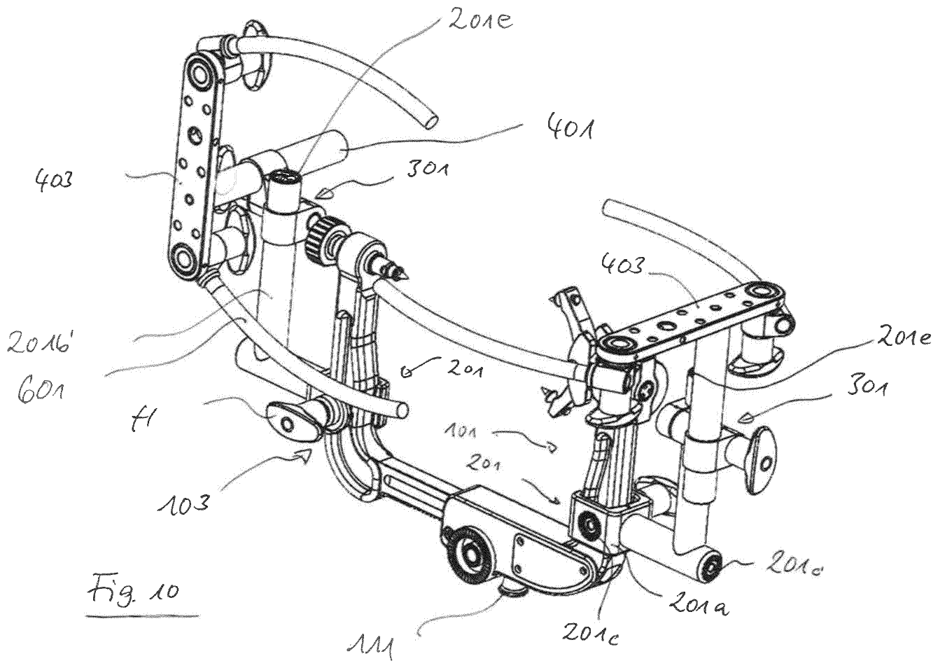

FIG. 10 shows a surgical head holding device according to the present invention in an exemplary third embodiment in perspective view from the top right;

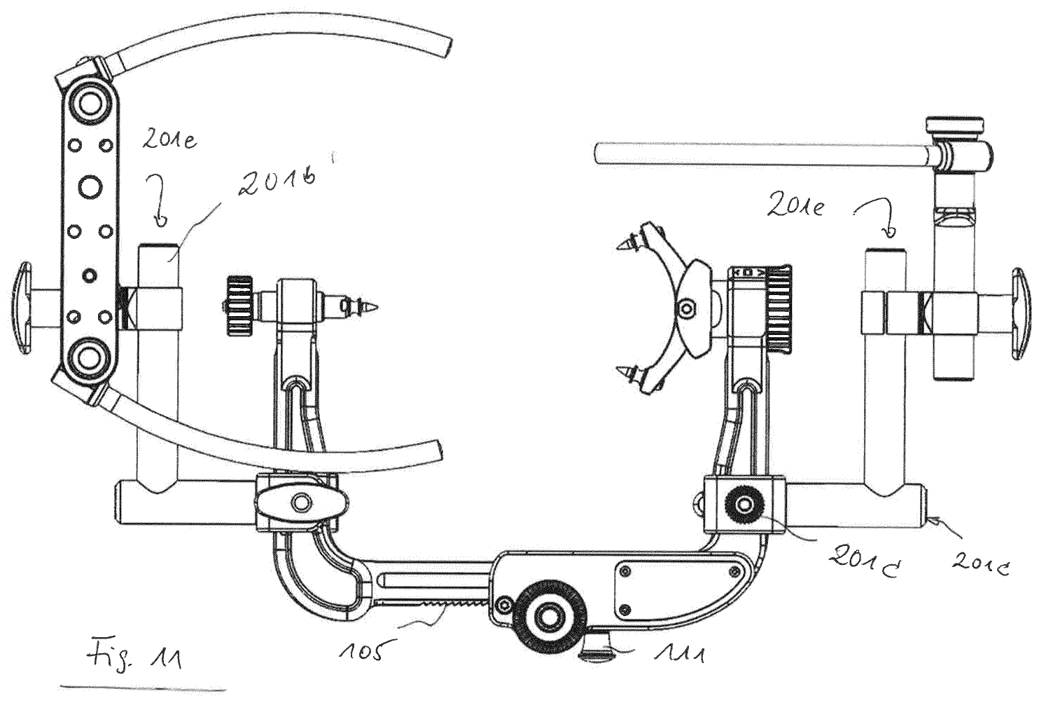

FIG. 11 shows the head holding device of FIG. 10 from the side;

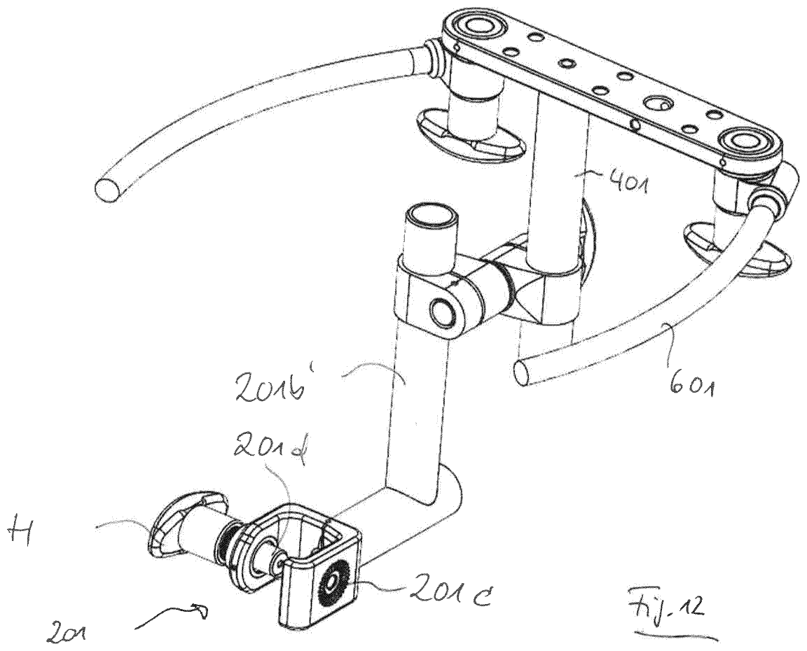

FIG. 12 shows the structure connected to the second arm in FIG. 10 in perspective;

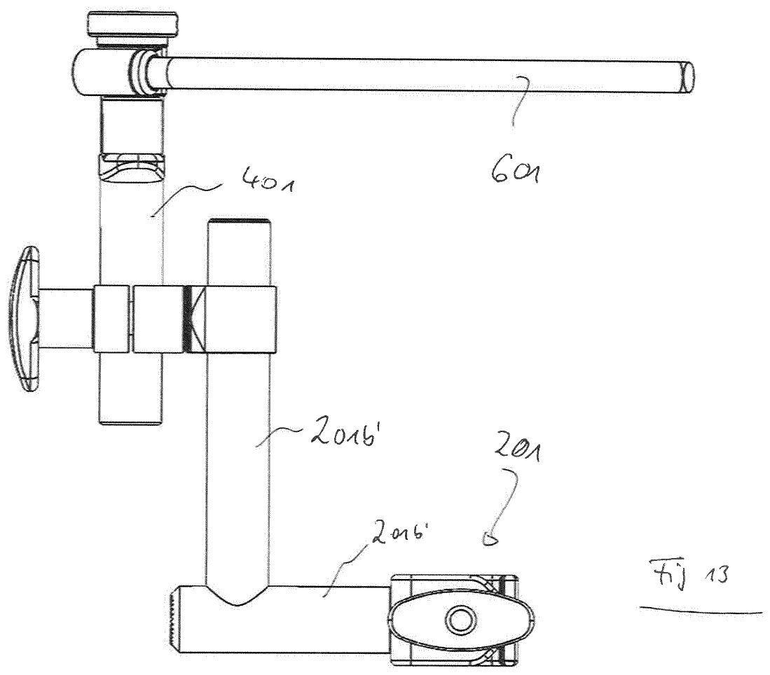

FIG. 13 shows the structure shown in FIG. 12 from the side;

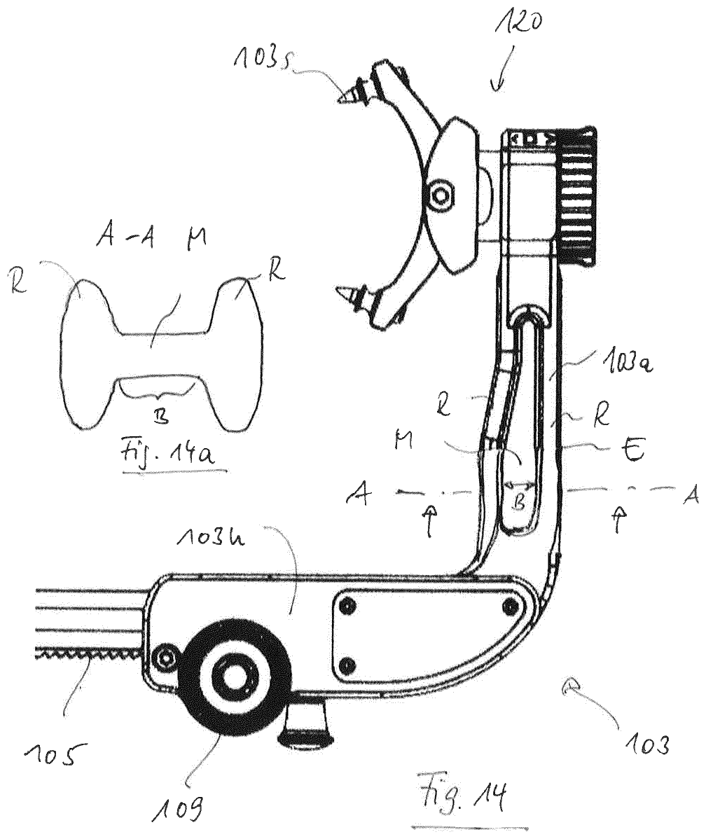

FIG. 14 shows the second arm of the head holding device of any of the preceding figures in view of its front side; and

FIG. 14a shows a cut A-A from FIG. 14

FIG. 1 shows an exemplary surgical head holding device 1000 according to the present invention, in slight perspective from above.

The head holding device 1000 has a head clamp 100 with a first arm 101 and a second arm 103.

The first arm 101 comprises a horizontal section 101h and a section 101a ascending therefrom (with respect to its arrangement in FIG. 1).

Likewise, the second arm 103 comprises a horizontal section 103h and a section 103a ascending therefrom (with respect to its arrangement in FIG. 1).

The ascending section 101a, 103a may alternatively be described as “protruding” or “standing away”.

The horizontal sections 101h and 103h may be connected to each other in a non-releasable or releasable manner. In each of these cases, they may be connected to one another in order to be adjustable relative to each other. Thus, as in the example in FIG. 1, a toothed rail 105, a ratchet, or the like may be provided.

One, two or more gear rims 107, 109 may be provided for attaching accessories or instruments to the head clamp 100, e.g. on one of the horizontal sections, as shown here for the horizontal section 101h. Other interfaces or docking points for attaching accessories or instruments to the head clamp 100 may be provided, for example, at horizontal sections 101h and 103h or ascending sections 101a and 103a. They may for this purpose be provided integrally or connected (screwed, welded, etc.).

Both the first arm 101 and the second arm 103 each carry, using their ascending sections 101a and 103a, at least one pin for clamping the skull between them, wherein the ascending section 101a herein exemplarily carries two pins 101s and the ascending section 103a carries one pin 103s.

The first arm 101 is connected to a clamp 201 in its ascending section 101a, which is represented in more detail in FIG. 7.

The clamp 201 may optionally be provided to be shifted along the ascending section 101a. The range in which a shiftable is possible may optionally be limited, for example by webs, steps, the choice of the diameter or another dimension of the arm 101 at this point, etc.

The clamp 201 may comprise a clamping shoe 201a and a protrusion 201b being optionally connected thereto in a rotation-proof manner or integral therewith. In FIG. 1 the protrusion 201b is exemplarily designed as a cylindrical section.

The protrusion 201b or another section of the clamp 201 may comprise an interface or coupling point for connecting an instrument or accessory, e.g. in the form of a gear rim 201c.

A first connection arrangement 301, which in this example comprises three through-openings, of which, optionally exactly, two comprise a variable inner diameter, is releasably clamped in one of these through-openings on projection 201b. As long as it is not clamped, the first connection arrangement 301 is rotatable around the protrusion 201b. Tightening a clamping screw 301a, e.g. by a screw handle or handle H, clamps the first connection arrangement 301 with the protrusion 201b or against it.

A first bar 401 is inserted into a further through-opening with a variable diameter of the first connection arrangement 301. It may also be rotated within the through-opening receiving it and/or may be displaced in its height in relation to the first connection arrangement 301 as long as the clamping screw 301a has not been tightened or this has not been prevented in some other way.

As shown in FIG. 1, the first connection arrangement 301 thus defines a first axis of rotation, which in relation to FIG. 1 corresponds to the longitudinal axis of the protrusion 201b, and a second longitudinal axis which corresponds to the longitudinal axis L of the first bar 401 being shown as a dash-dotted line.

A third axis of rotation, which is defined by the first connection arrangement 301, is made possible by a twisting arrangement, which is here optionally designed as a combination of two gear rims 301c, as may optionally also be present on other connection arrangements shown in FIG. 1. It corresponds in FIG. 1 to the longitudinal axis of the clamping screw 301a, or is parallel to it.

The first bar 401 comprises a first end section 401a (bottom of FIG. 1) and a second end section 401b (top of FIG. 1).

The first end section 401a is here exemplarily a free end, which optionally carries a radially protruding locking pin 401c.

The locking pin 401c may ensure that the first bar 401, which is rotatable within the first connection arrangement 301 in the unclamped state, can only be pulled out of the through-opening of the first connection arrangement 301 at a small angle of rotation along its longitudinal direction, since the locking pin 401c can only be passed through the through-opening together with the first bar 401 in the area of a clamp opening 301d.

Locking pins similar to locking pin 401c may optionally be provided on any of the rods described herein.

The first bar 401 comprises a rail 403. Here, it is optionally connected to the second end section 401b or manufactured integrally therewith.

The rail 403 can be welded to the first bar 401, integrally manufactured or otherwise connected in a rotationally fixed, non-releasable and/or form-fit and/or force-fit manner. If, as in the present example, the possibility of rotation between bar 401 and rail 403 is dispensed with, a mechanism for allowing and preventing rotation may also be dispensed with, which may have the advantage that protrusions and instabilities that would be caused by such a mechanism do not have to be provided nor occur at this prominent location where the surgeon works.

The rail 403 can form a T-shape with the first bar 401. The rail 403 may have two opposite ends 403a and 403b, at which the rail 403 ends freely.

The rail 403 may have openings 403c or through-openings 405 for fastening additional devices or instruments. These may be provided on and/or accessible from any surface of the rail 403.

For manufacturing and cost reasons, the rail 403 may have a straight course, optionally not a curved course. If the rail 403 has a curved or otherwise non-straight course, it is advantageously convex to the head, i.e. its ends are farther from a center of the head clamp 100 than a central section of the rail 403. The straight course, like the course of the rail 403 curved or angled away from the center, allows the largest possible free space between rods in which the surgeon can find space for surgery.

The rail 403 has coupling points to which accessories or instruments may be attached directly or indirectly.

These coupling points include through-openings 405, which may be provided at one or both ends 403a and 403b, and which, e.g. may be equipped with a thread.

A second connection arrangement 501 is connected to the rail 403 at the left end of the latter, here, exemplarily, by its left through-opening 405. A similar or identical connection arrangement 501 is connected to the right end of the rail 403, here exemplarily by its right through-opening 405.

The second connection arrangements 501 in turn have a clamping screw which may be tightened and released again by a handle. Other locking mechanisms which do not act by a screw connection, but use e.g. toggle levers, eccentrics or similar, are also being considered.

The second connection arrangement 501 comprises an insertion opening 501a into which a bracket 601 is inserted. The bracket 601 may be pivoted by the second connection arrangement 501 in one plane, in the settings of FIG. 1 exemplarily in the horizontal plane. The axis of rotation about which this pivoting takes place is defined by the second connection arrangement 501 and corresponds in the example of FIG. 1 to the longitudinal axis of the clamping screw, the free end 503 of which more or less ends or closes with the upper surface of the rail 403.

In the embodiment shown in FIG. 1, the second connection arrangement 501 allows the bracket 601 to be rotated about exactly one axis of rotation, namely the one discussed above. For this purpose, the bracket 601 can be inserted into the second connection arrangement 501 in a rotation-proof or form-fit manner.

The bracket 601 may in turn be used to attach instruments, hooks, spreaders or the like. Its profile may be circular, ring-shaped or of a different shape.

FIG. 1 shows an optional further connection arrangement 501 of the above-mentioned type, which is also connected to the above-mentioned rail 403.

The above description of FIG. 1 has been limited to structures or additions which are connected to the first arm 101 or are supported by the latter.

Descriptions of elements that are connected to the first arm 101 apply analogously also to the same or to similar elements that are connected to the second arm 103, which can be seen not least from the figures. In order to avoid repetitions, their description is omitted. Instead, reference is made to the description of such elements with reference to the first arm 101.

The following description of FIG. 1 deals with structures or attachments which are connected to the second arm 103 or are supported on the latter. In this, hereinafter, those elements that differ from elements that are described in connection with the first arm 101 will be discussed.

The second arm 103 with its ascending section 103a is connected also to a clamp 201, as already discussed above. A first connection arrangement 301, as already also discussed above, is connected to the clamp 201.

In the first connection arrangement 301 there is a second bar 402 optionally comprising a tongue 402d which prevents the second bar 402 from rotating in the first connection arrangement 301. It is pointed out, however, that this tongue 402d is optional as well as other anti-rotation mechanisms (non-round cross-section, etc.) and that the first bar 401 could also comprise such a tongue.

A groove which together with the tongue 402d forms an anti-rotation protection for the second bar 402 in the first connection arrangement 301, may be provided in the first connection arrangement 301. Alternatively, the tongue may be provided in the first connection arrangement 301, but the groove may be provided on the second bar 402. Also alternatively, another anti-rotation protection may be provided instead of a tongue-groove system. However, any kind of anti-rotation protection is always optional.

As FIG. 1 shows, the second bar 402 with a further end section thereof, which optionally carries at the front end an interface, e.g. a gear rim 402e, is inserted in a further connection arrangement 701, herein referred to as the third connection arrangement. The third connection arrangement 701 may be designed like the second connection arrangement 501.

The third connection arrangement 701 connects the second bar 402 to a further first bar 401. Like the second bar 402, the first bar 401 may optionally comprise an anti-rotation protection, for example, in the form of a tongue 401d, a groove, or the like.

Just like the first bar 401 which is described with reference to the first arm 101, here the first bar 401 may be connected to a rail 403 too, as described above, or may be manufactured integrally.

The first connection arrangement 301, which is connected to the second arm 103 as well as to the second bar 402 by the clamp 201, preferably allows or defines a rotation around exactly two axes of rotation.

The third connection arrangement 701, to which the second bar 402 is also connected, preferably allows or defines a rotation about exactly one axis of rotation.

FIG. 2 shows the surgical head holding device 1000 of FIG. 1 from the side.

Handles used to tighten or lock clamping screws are marked with the reference sign H in FIG. 2.

FIG. 3 shows, in perspective, the first rod 401 of FIG. 1 together with the rail 403 and two brackets 601 connected to the rail 403 by two second connection arrangements 501.

FIG. 4 shows what is shown in FIG. 3 from the front.

It can be seen that the rail 403 does not have to sit centrally above or over the first bar 401, although this is also possible according to the present invention. Thus, the T-shape mentioned herein may also be a T with unequal legs.

FIG. 5 shows an alternative embodiment of the one shown in FIG. 3.

Unlike in the embodiment of FIG. 3, the brackets 601 (one or both) are not curved, but rather straight in their longitudinal extension.

The bracket 601 of FIG. 5, one or both, may have a round diameter at least in sections. This is optional.

As in FIG. 4, the rail is not arranged with the same projection to the left or to right above the first bar 401.

FIG. 6 shows what is shown in FIG. 3 from above.

The arrows indicate the (only) direction of rotation in which the bracket 601 may rotate about an axis of rotation in the second connection arrangement 501, said axis of rotation is designated by a cross X. Rotation of, for example, an insertion portion 601a of the bracket 601 within the second connection arrangement 501 may—optionally irrespective of whether or not the connection arrangement 501 is tightened by the handle H and the clamping screw connected thereto—be impossible, for example due to welding the bracket 601 in the second connection arrangement 501, due to a tongue and groove system or the like. This design advantageously allows a high load on the bracket, in particular by a torque, for example by attachments provided on bracket 601.

FIG. 7 shows, in perspective, the structure connected to the second arm 103 in FIG. 1, but without the arm 103.

The focus of attention in FIG. 7 is on the exemplary design of the clamp 201. Its clamp shoe 201a, which here may optionally include the handle H and the clamping screw 201d connected thereto, may be designed as a U-profile in this embodiment—or in any other—according to the present invention. The U-profile does not require claws, but surrounds a section of the arm, especially the ascending one.

FIG. 8 shows sections of what is shown in FIG. 7 from the front and slightly from above.

FIG. 9 shows a surgical head holding device 1000 according to the present invention in an exemplary second embodiment in a perspective view from the top right.

This embodiment corresponds essentially to that of FIG. 1. Only the differences from the embodiment of FIG. 1 are to be mentioned below. They only relate to the structure that is supported on the second arm 103.

Starting from a clamping shoe 201a of the clamp 201, as is also known from FIG. 1, the protrusion 201b′ extends in a form-fit manner. Unlike the protrusion 201b in FIG. 1, however, this is not designed as a straight section, but has both a horizontal section (in relation to FIG. 9) and a section ascending from it, which, in FIG. 9, runs vertically. The section referred to herein as horizontal and the section referred to herein as ascending may be perpendicular to each other, they may be at a different angle to each other, but they are not parallel and do not have a common longitudinal axis.

A joint may be provided between the horizontal section and the ascending section. Preferred, however, there is no joint. The horizontal section and the ascending section are optionally made in one piece or integrally. Alternatively, they are welded together.

The first connection arrangement 301 in turn connects the protrusion 201b′ to the first bar 401 in a manner known from FIG. 1 and also shown in FIG. 9 for the first arm 101.

A gear rim 201eor another interface may optionally be arranged on the clamp 201 on the end face or at an upper end thereof, for example for coupling further accessories, such as further holders, positioning devices and/or instruments, etc.

A groove-tongue connection or other anti-rotation protection on the first bar 401 may be provided, but is dispensable.

A pin lock on the first bar 401 for protection against unwanted pulling out may be provided, but is not shown in FIG. 9. Such a securing against pulling out, which may be designed as a pin lock, may be optionally provided here as well as on any other bar mentioned herein.

The protrusion 201b′ may at any site have at least one interface or coupling point, for example designed as a gear rim 201c. This is optional

FIG. 10 shows a surgical head holding device 1000 according to the present invention in an exemplary third embodiment in perspective view from the top right.

This embodiment corresponds in many aspects to that of FIG. 9. In deviation from that, however, both arms 101 and 103 are here connected with a combination of a clamp 201 with a protrusion 201b′ and a first bar 401, as is shown in FIG. 9 only with reference to the second arm 103.

One of the clamps 201 shown in FIG. 10 is mounted with handle H pointing forward (left in FIG. 10), the other with handle H pointing backward. It can be seen on the latter that the clamp shoe 201a may also have a gear rim 201d or a differently designed interface, i.e. not only the protrusion 201b.

The rail 403 shown on the left in FIG. 10 is inclined by 90° compared to the rail 403 shown on the right. This is made possible by the first connection device or connection arrangement 301.

It can be seen from the inclined left rail 403 that the protrusion 201b′ may have an interface such as a gear rim 201c not only in its section shown horizontally in FIG. 10, but may also have such an interface, here gear rim 201e, in its section that extends away from the section shown horizontally at an angle (which is vertical here). In this way, the clamp 201 allows an accessory to be connected also from above, not only in a horizontal direction. In addition, this may advantageously increase the number of interfaces. Providing a number of interfaces as high as possible on the clamp 201 may also offer the advantage that a large number of accessories or instruments may also be adjusted in height together with the clamp 201, which is optionally height-adjustable along the arm. This would be different if the relevant interface would be attached for example to the arm itself, and thus always at the same height above the horizontal section.

Advantageously, the height adjustability of the interfaces of the clamp 201 with respect to, for example, the operating table may omit the need for height adjustment mechanisms of the instruments or accessories connected to one of the arms 101, 103.

Thus, the clamp 201 may comprise a plurality of interfaces, e.g. gear rims, that enables coupling of instruments or accessories preferably from more than one direction and, in their entirety, preferably in more than one plane.

A spring-mounted button serves as an example of a securing device 111 to secure the gear rack 105 against sliding movement by engaging the teeth of the gear rack with protrusions, teeth or the like. When or if it is moved or retracted against the spring action, it gives up its clamping or locking action, and the two horizontal sections 101h and 103h may be moved relative to each other to adjust the head clamp 100 to the patient to be treated, thereby changing the distance between the pins 101s and 103s.

FIG. 11 shows the head holding device of FIG. 10 from the side.

FIG. 12 shows the structure connected to the second arm in FIG. 10 in perspective.

FIG. 13 shows the structure shown in FIG. 12 from the side.

FIG. 14 shows the second arm 103 with a view of its front side, but without the clamp 201, which allows a view of the profile of the ascending section 103a.

A cut through this section is shown in FIG. 14a. The profile shown has the shape of a double T or an H with a middle section M and two edge webs R delimiting this middle section M in the longitudinal course of the ascending section 103a. The edge webs R protrude beyond a level of the middle section M. In other words, the middle section M is set back in relation to the edge webs R adjacent to it, namely on optionally both the front side and on the rear side of the ascending section 103a.

As in the example of FIG. 14, the width B of the middle section M optionally changes with increasing distance from the horizontal section 103h. In the example of FIG. 14, it tapers toward the top, and it becomes wider toward the bottom.

Optionally, however, at least one section is also provided in which the width B is constant. This section is optionally at least 1, 2 or 3 cm long.

The width B may be dimensioned such that the tip of the clamp screw 201d (or an element placed on the free end thereof, not shown in FIG. 14) may find space between the edge webs R at selected height portions of the ascending section 103a, somewhat higher however not more, what cannot be ignored by the user or absolutely prevents the clamp 201 from being attached to the ascending section 103a.

This design advantageously serves to prevent wanting to attach the clamp at a height of the ascending section 103a at which the clamp 201 must not be attached. There can be many reasons why the clamp 201 must not be attached beyond a predetermined end height, denoted by E in FIG. 14. For example, it can be determined that a minimum distance must be maintained between the location at which the clamp 201 is placed and that of a tip holding arrangement 120. Similarly, if the overall upwardly tapering ascending section 103a exceeds a certain height above the horizontal section 103h, the arm may no longer have the necessary strength to safely support the clamp 201 together with the forces acting thereon during use. Or, the ascending section 103a, which tapers as a whole, is not wide enough from a certain height to be able to rest against opposing walls on both sides inside the clamping shoe 201a and to be able to be supported thereon.

List of reference numerals

| Reference | |
|---|---|
| numeral | Description |
| 1000 | head holding device |
| 100 | head clamp |
| 101 | first arm |
| 101h | horizontal section of the first arm 101 |
| 101a | ascending section of the first arm 101 |
| 101s | pin |
| 103 | second arm |
| 103h | horizontal section of the second arm 103 |
| 103a | ascending section of the second arm 103 |
| 103s | pin |
| 105 | gear rack |
| 107 | gear rim |
| 109 | gear rim |
| 111 | securing device |
| 120 | tip retaining or holding arrangement |
| 201 | clamp |
| 201a | clamp shoe |
| 201b | protrusion |
| 201c | gear rim |
| 201d | clamp screw |
| 201e | gear rim |
| 201b′ | protrusion |
| 301 | first connection arrangement |
| 301a | clamping screw |
| 301c | combination of two gear rims |
| 301d | clamp opening |
| 401 | first bar |
| 401a | end section |
| 401b | end section |
| 401c | locking pin |
| 401d | tongue |
| 402 | second bar |
| 402d | tongue |
| 402e | gear rim |
| 403 | rail |
| 403a | end |
| 403b | end |
| 403c | openings |
| 405 | through-opening |
| 501 | second connection arrangement |
| 501a | insertion opening |
| 503 | free end of the clamp screw |
| 601 | bracket |
| 601a | insertion opening |
| 701 | third connection arrangement |
| B | width |
| E | end height |
| H | handle |
| L | longitudinal axis of the first bar |
| M | middle section |
| R | edge webs |

1. A surgical head holding device for use in a medical treatment, wherein the surgical head holding device encompasses:
a head clamp having a first arm and a second arm, wherein both the first arm and the second arm have each at least one pin for receiving or clamping the head therebetween;
a first bar which comprises a rail extending radially and/or perpendicular to the longitudinal axis of the first bar; and
a first connection arrangement for connecting the first bar to the head clamp.

2. The surgical head holding device according to claim 1, wherein:
the first connection arrangement is a clamping device.

3. The surgical head holding device according to claim 1, further comprising:
a bracket; and
a second connection arrangement configured to connect the bracket to the rail.

4. The surgical head holding device according to claim 1,
wherein the first connection arrangement or the second connection arrangement is designed to define one first axis of rotation and to enable a bar or bracket received therein to use this connection arrangement exclusively to rotate about the first axis of rotation, and
wherein the other of these two connection arrangements is designed to define at least one second axis of rotation and one third axis of rotation and to enable a bar or bracket received therein to rotate about at least both said second axis of rotation and said third axis of rotation by this connecting arrangement.

5. The surgical head holding device according to claim 1, further comprising:
a second bar; and
a third connection arrangement configured to connect the second bar to the first bar.

6. The surgical head holding device according to claim 5, wherein the third connection arrangement is configured to define one fourth axis of rotation and to enable a bar received therein to exclusively rotate about the fourth axis of rotation by this connection arrangement.

7. The surgical head holding device according to claim 1, wherein the first connection arrangement comprises or is connected to a clamp for connecting the first connection arrangement to the first arm.

8. The surgical head holding device according to claim 7, wherein the clamp is shiftable along the first arm.

9. The surgical head holding device according to claim 7, wherein the clamp comprises a threaded screw, but does not comprise claws.

10. A bar which comprises, or is connected to, a rail extending radially and/or perpendicularly to the longitudinal axis of the first bar, for the connection to a head clamp for or of a surgical head holding device.

11. The surgical head holding device according to claim 1, wherein the rail is at an end section of the first bar.

12. The surgical head holding device according to claim 7, wherein the clamp is for connecting the first connection arrangement to the section of the first arm carrying the at least one pin.

13. The bar according to claim 10, wherein the rail is at or connected to an end section of the bar.
